(19) United States
(12) Patent Application Publication
(10) Pub. No.: US 2024/0407526 A1
(43) Pub. Date: Dec. 12, 2024

(54) A FINISHING PROCESS FOR COATING A SURFACE OF A SOLID COSMETIC PRODUCT WITH PIGMENTED PARTICULATE MATERIAL

(71) Applicant: GOTHA COSMETICS S.R.L., Lallio (IT)

(72) Inventors: Marcelo SANTILLAN, Lallio (IT); Luca DALLATORRE, Lallio (IT)

(21) Appl. No.: 18/699,190

(22) PCT Filed: Oct. 7, 2022
(86) PCT No.: PCT/IB2022/059596

§ 371 Date: Apr. 5, 2024

(30) Foreign Application Priority Data
Oct. 8, 2021 (IT) 102021000025808

(51) Int. Cl. A45D 33/18

(52) U.S. Cl. CPC A45D 33/18

(57) ABSTRACT

The present invention relates to a process for coating a surface of a compact solid cosmetic product obtained by casting with pigmented particulate material.

The present invention concerns a process for completing a cosmetic product with special surface effects and the cosmetic products thus obtained. In particular, the present invention relates to a process for producing special surface effects on cosmetics of the family of anhydrous cosmetics, more in particular of a compact solid cosmetic product obtained by casting (for the sake of simplicity hereinafter defined only as solid cosmetic product), such as the poured cosmetic product. More in particular, the present invention refers to a completion (or finishing) process of a surface of a solid cosmetic product finished with a pigmented particulate material, in particular coloured pearls.

As mentioned above, the process according to the present invention is applied in the field of the production of compact solid cosmetic products obtained by casting, for example blush/rouge, face powder, concealer, foundation, lip gloss, primer, eyeshadow, etc. Said type of product, once finished, is in solid state, and therefore may or may not be provided with a secondary packaging. Said secondary packaging usually forms a support, also called pan, on which the cosmetic product itself rests.

The aforesaid special surface effects are meant, for example, as patterns, colour nuances, decorations, etc. Hereinafter, the generic term “decoration or “decorations” shall mean any type of pattern, colour effect, etc.

There are known decoration methods through which the finished solid cosmetic product is decorated by tracing patterns, writing or the like on its surface through the use of powder or fluid products, These methods allows colour contrasts to be produced and create an aesthetic effect.

One of these methods provides for spraying a liquid coloured product on the surface of a finished cosmetic product, making the flow of sprayed product pass through coloured masks.

This system is not optimal as it produces surfaces and/or decorations that are not well defined and, moreover, a large portion of this product is deposited on “non-useful” surfaces, i.e., different from the desired surfaces of the cosmetic product, such as the surfaces surrounding the surface concerned, including, for example, the external surface of the walls of the pan, in particular the rim defining an opening present in the pan. This deposit on non-useful surfaces leads to a high waste of liquid coloured product, and, consequently, represents a considerable drawback on an industrial scale due to economic losses, both direct and indirect, such as for cleaning costs.

The Applicant has now surprisingly found a new completion process that allows decorations having particular visual effects to be obtained on the external surface of a compact solid cosmetic product obtained by casting, for example, such as a cosmetic product in a pod. In particular, a decoration that has a velvety, or also three-dimensional, visual effect can be produced.

This visual effect is achieved thanks to the use of pigmented particulate material not carried by a liquid component; this was made possible due to the development of a specific process conducted using pigmented particulate material in powder state, rather than transported by a liquid carrier, as occurs in some completion processes known in the art. Moreover, the process according to the present invention overcomes the aforesaid problems related to the loss of product following deposit on said non-useful surfaces and the need to clean the solid cosmetic product from at least some surfaces, intended in a broad sense, such as the pan when it is present. In fact, with the completion process according to the present invention it is possible to obtain a cosmetic product (whether considered per se or also with its secondary packaging) that has the pigmented particulate material, at the end of the deposition step of this material, deposited solely on the surface intended to be coated with this material, i.e., the surface of the cosmetic product exposed to the consumer's view, i.e., facing the consumer (for brevity hereinafter also defined as external surface or surface concerned by the cosmetic product), and not also in other areas of the cosmetic product. For example, in the case of cosmetic product in pod form, on the lateral surface of the pod or on the surface of the pan (typically on its rim).

Keeping said surfaces free of the pigmented particulate material is advantageous for various reasons. In fact, the pigmented particulate material deposited on said surface has no decorative purpose, and moreover could also be counterproductive and thus require to be removed. By remaining on these surfaces, said material would, at the very least, be wasted, as in the case in which said parts are concealed from the consumer's view, once the cosmetic product has been packaged in its secondary or primary container. At least on some of these surfaces said material can also have a detrimental effect, above all if this surface is the surface of the pan. For example, in particular, if said material is present on the pan, it gives an unpleasant sense of dirtiness and, moreover, the presence of pigmented particulate material on the pan usually exacerbates the adhesive properties of the pan (with respect to the same surface without said pigmented particulate material) to the wall of the case (i.e., the primary packaging) into which the pan will subsequently be inserted. Therefore, following the presence of this pigmented material on said surfaces, in particular the surface of the pan, if present, cleaning will be necessary. With the process according to the present invention, said surfaces will not be concerned by deposition of the pigmented particulate material resulting in a consumption of less pigmented particulate material and, possibly, omission of the step to clean of said surfaces.

A first economic advantage offered by the process according to the present invention is that this process allows the use of smaller amounts of material for decorative use, i.e., the pigmented particulate material, for each single decoration to be made with respect to what occurs with processes known in the art. In fact, in the process according to the present invention, the pigmented particulate material is used in an amount of around 20-30% less by weight with respect to the amounts needed with processes that require the pigmented particulate material in a liquid carrier.

A second advantage obtained by the present process is the possibility of recovering the pigmented particulate material that has not deposited on the external surface of the solid cosmetic product itself. Said recovered material is not altered, and, therefore, can be used again in a subsequent completion process.

Therefore, the subject matter of the present invention is a completion process of a compact solid cosmetic product obtained by casting, said product having a surface to be completed intended to be coated with pigmented particulate material, said process comprising the following steps:
I) positioning a mask on said surface to be completed of the compact solid cosmetic product obtained by casting, said mask being suitably perforated so as to leave at least a portion of said surface of said cosmetic product exposed;
II) dispersing a pigmented particulate material on said exposed surface portion of surface to be completed of said cosmetic product, said material being free from liquid carrier;
III) suctioning the pigmented particulate material deposited on surfaces different from said exposed surface portion to be completed of said cosmetic product; and
IV) collecting the pigmented particulate material not deposited on the cosmetic product.

In the process according to the present invention said steps follow one another in the aforesaid order.

The compact solid cosmetic product obtained by casting to be subjected to the process according to the present invention is a solid body that can have any form, typically, but not only, pod form. This body typically has an external surface intended to be completed by coating with said pigmented particulate material. Said external surface intended to be completed (hereinafter, for brevity, indicated only as external surface) of the cosmetic product to be completed is the one that will be visible to the consumer.

The whole of the external surface, or only a portion thereof, can be coated with the pigmented particulate material.

The external surface to be completed can be flat, concave, convex or undulated.

Moreover, this external surface can be smooth or rough, for example as a function of the material with which the cosmetic product or the counter-mould is prepared. A smooth surface can, possibly, be processed so as to make it slightly rough.

The groove has a height and a distance from the adjacent groove that are functional to various parameters, mainly to the decoration to be reproduced on the external surface, to the marked or subtle effect desired and to the particle size of the pigmented particulate material.

If present, the grooves that are created have an indicative height ranging from 0.001 to 45 mm.

In said process, the pigmented particulate material is, for example, of pearlescent type.

The finished solid cosmetic product to be completed can be provided with a secondary 15 packaging that acts as support, as previously specified also called pan, or can be without said secondary packaging. Hereinafter, unless otherwise specified, the terms secondary packaging, support and pan are used in interchangeably.

In the first case, the process according to the present invention is preceded by a filling step (hereinafter step R), which consists of filling said pan (provided with an opening) with the corresponding semi-finished cosmetic product. Filling of the pan takes place by pouring the semi-finished cosmetic product.

The process comprising said step R forms a preferred embodiment, although this step R can be omitted in the case of cosmetic products not obtained, for example, by pouring.

Therefore, below the process according to the present invention is described as comprising said step R, and consequently the pan is referred to. 25

In this step R, the semi-finished cosmetic product (hereinafter product PS) is poured into a pan. The product PS is in molten form, generally fluid or semi-fluid. Filling can take place from above or from the rear, according to the type of pouring performed. In the case of a pan filled by pouring from the rear, the pan is filled to its rim.

The pan used that is to be filled with the product PS has any shape, typically circular.

In general, the pan has a bottom and side walls that define an opening. This opening is opposite the bottom, and generally has approximately the same dimensions as the bottom.

Once the product P has been introduced into pan and has filled it (completely or not according to the type of pouring performed) and, possibly, is distributed therein, the product PS is cooled to room temperature (i.e., around 20-25° C.).

At the end of this step a finished cosmetic product (hereinafter product PF) in a secondary packaging is obtained. Once this product PF has been inserted into the pan it has an external surface, i.e., the surface not surrounded by the walls of the pan. Said external surface will be the surface completed with the decoration implemented according to the process according to the present invention.

Said product PF thus provided with secondary packaging is ready to be subjected to the actual completion process, which starts with said step I.

In the present text, external surface of the product PF is meant as the surface not in contact with the wall or with the bottom of the pan.

Before starting step I, the product PF to be completed is, preferably, placed in a suitable container, called pallet. This container can have many forms also according to whether the product PF to be completed is in a pan or has no support.

The pallet can have a bottom with, for example, two or more recesses into which to insert one pan in each recess. Moreover, or alternatively, the pallet is provided with a grid placed inside so as to cover the internal surface of the bottom of the pallet. The grid is intended to serve as a support base for the product PF to be completed.

According to another possible variant, the pallet has a bottom from which the walls of the pallet or isolated supports rise. Said walls or supports form the support base for the mask. In this variant, the mask has more than one perforation, each corresponding to each product PF to be completed without secondary packaging.

In step I the external surface to be completed of the product PF is covered with a specific perforated mask.

The perforation, in the form of through cuts, on the mask represents the decorations to be reproduced on the external surface of the product PF to be completed. The perforation leaves the portion of the external surface of the cosmetic product below exposed, this portion of the external surface can thus be coated with the pigmented particulate material released during the deposition step of the process according to the present invention.

Said mask (also known as stencil or silhouette) used for this purpose is of the type known to the person skilled in the art and consequently will not be further described.

Generally, the mask rests on the walls that define said opening of the pan, typically on the rim of these walls, if the pan is present, or on the pallet, typically on the rim of these walls, if the product PF has no pan.

Preferably, the mask is placed so that it is not in contact with the external surface of the cosmetic product to be completed, generally the distance between said surface and the mask varies, indicatively from 0.01 to 15 mm.

The mask can, if necessary, be provided with feet, if necessary adjustable, to ensure said distance between it and said external surface.

Subsequently to said step I, the finished cosmetic product covered with the mask is positioned in an area for depositing the pigmented particulate material, in which the deposition step of pigmented particulate material on the external surface of the cosmetic product is performed (step II or spraying step).

According to an aspect of the invention, this step provides for dispersing the pigmented particulate material in the air and its deposition by fall or precipitation on said external surface of the cosmetic product.

This area comprises at least one dispersing device that delivers the pigmented particulate material to be deposited on the external surface of the cosmetic product to be completed.

Naturally, the area for depositing the pigmented particulate material can comprise a certain number of dispersing devices, indicatively from 1 to 20, according to the decoration to be applied on said surface.

The dispersing device used in the process according to the present invention is, typically, an auger or a mechanical dispersing device.

The capacity of the dispersing device and the speed of delivery of the pigmented particulate material by the dispersing device depend on the type of pigmented particulate material delivered.

The dispersing device has nozzles from which the pigmented particulate material exits. Said nozzles have, indicatively, a diameter of between 0.2 and 50 mm.

The pigmented particulate material is delivered from the dispersing device at a pressure indicatively of between 2-10 bar, preferably 6-8 bar.

After being delivered from the dispersing device, said pigmented particulate material is dispersed into the air and then deposits on said external surface of the cosmetic product.

Without wishing to limit the present invention to any operating theory, deposition of the pigmented particulate material takes place by mechanical depositing due to its fall by gravity and adheres to this external surface of the cosmetic product.

The deposition operation has a duration, indicatively, of between 2 and 300 seconds. The duration is a function of the effect to be obtained and of the type of pigmented particulate material used.

The pigmented particulate material deposits on the exposed portion of the surface of the cosmetic product, which forms the surface concerned, but also on the surfaces surrounding said exposed portion (hereinafter defined surfaces not concerned) hence, for example, on the perforated mask and on the pallet. On the surfaces not concerned, the pigmented particulate material does not adhere, and therefore is easily removable therefrom by suitably calibrated suction, as described below.

The pigmented particulate material is, preferably, selected from pearls, preferably coated pearls, and glitter powder.

The pearls are typically based on natural mica or synthetic mica (synthetic fluorophlogopite), aluminium, aluminium borosilicate and ferric ferrocyanide. More specifically, indicatively, the pearls are composed of the type with mica, synthetic mica, calcium aluminium borosilicate, calcium sodium borosilicate, silica, calcium titanium borosilicate, CI 77163-bismuth oxychloride, CI 77400-copper powder, CI 77400-bronze powder.

The coated pearls have a coating of a material selected, generally, from those that are silicone-based.

Indicatively, the glitter powder consists of material of the type with cellulose acetate, glass oxide, fluorophlogopite, aluminium and polyethylene terephthalate with polyurethane-11.

The pigmented particulate material generally has dimensions ranging from 5 to 500 μm, preferably 15 to 250 μm.

The subsequent step (step III) to said spraying step II, consists in suctioning the pigmented particulate material that was deposited surfaces other than the exposed portion of surface of said cosmetic product. These surfaces are, for example, the surface of the perforated mask, of the wall of the pan and the surface on which the pan rests in the area for depositing said pigmented particulate material. This suctioned material is an excess amount delivered with respect to the amount of material effectively used to produce the desired decoration.

Suctioning takes place according to methods known to a person skilled in the art, and therefore will not be described in detail. Suctioning takes place with a suction device. The power and/or the flow at which the suctioning device operates is in correlation to the type of pigmented particulate material to be suctioned and to the material of which the cosmetic product subjected to the completion process according to the present invention is formed. For example, in the case of the use of calcium aluminium borosilicate, suctioning is carried out with a flow rate of 5-35 m³/hour.

The pigmented particulate material in excess that was removed can thus be collected (step IV) and stored in specific containers for subsequent re-use.

The product obtained at the end of step III is a completed product, ready to be sent to the packaging site, where the final operations will be carried out according to the prior art concerning packaging of the completed product in the primary packaging.

Further features and advantages of the present invention will be more apparent from the description of an example of embodiment as illustrated in the accompanying figures, wherein:

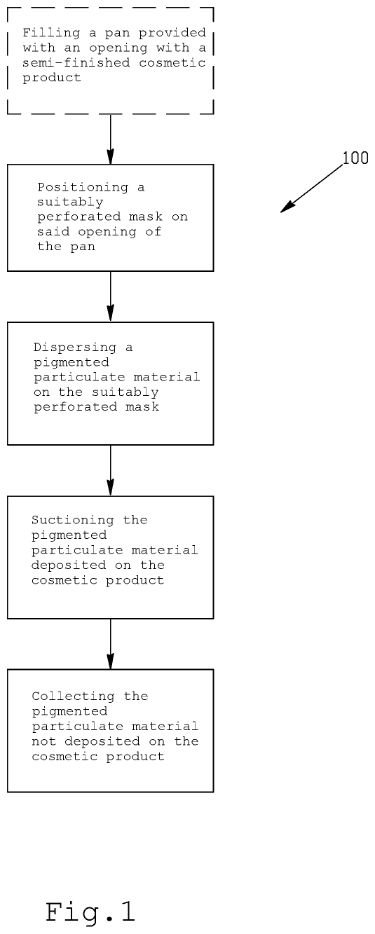

FIG. 1 is a representation of a block diagram of an embodiment of the process according to the present invention;

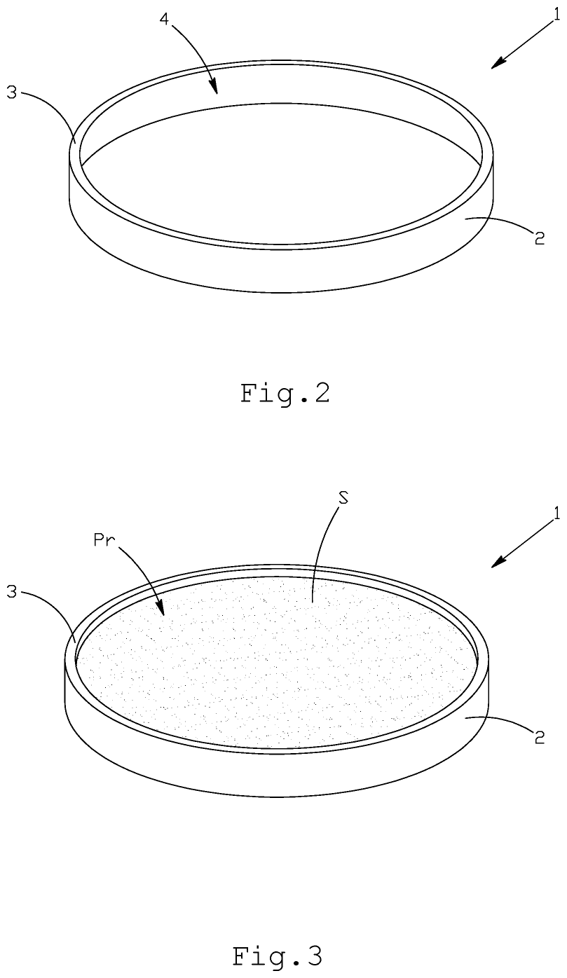

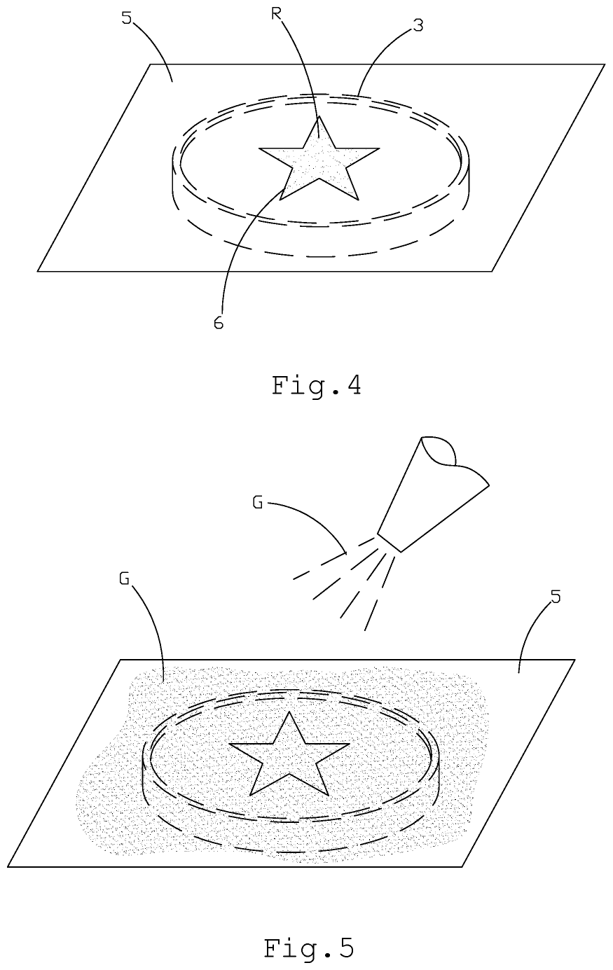

FIGS. 2 to 6 are axonometric views from above of a solid cosmetic product in a pan in different processing steps of the process according to the present invention;

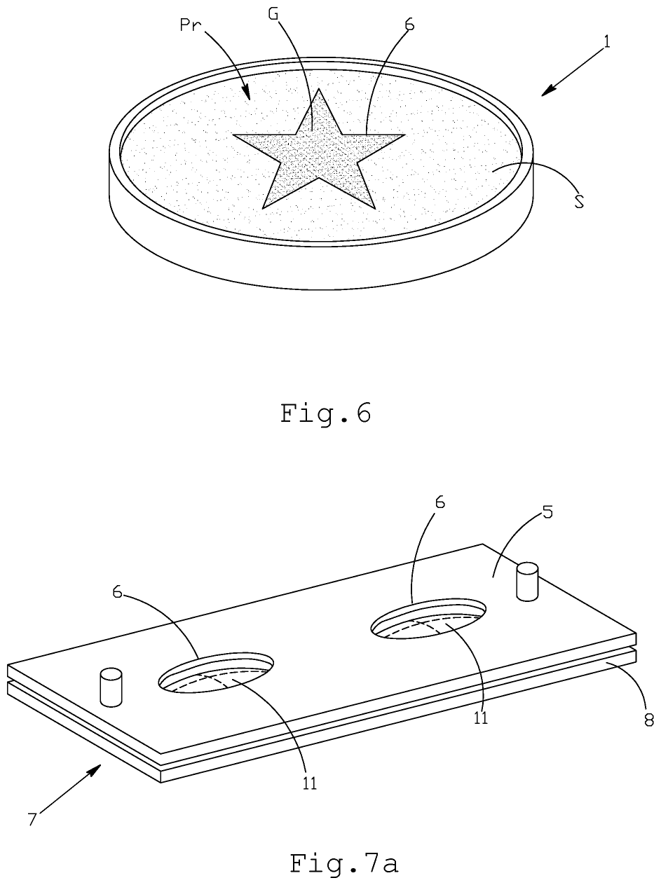

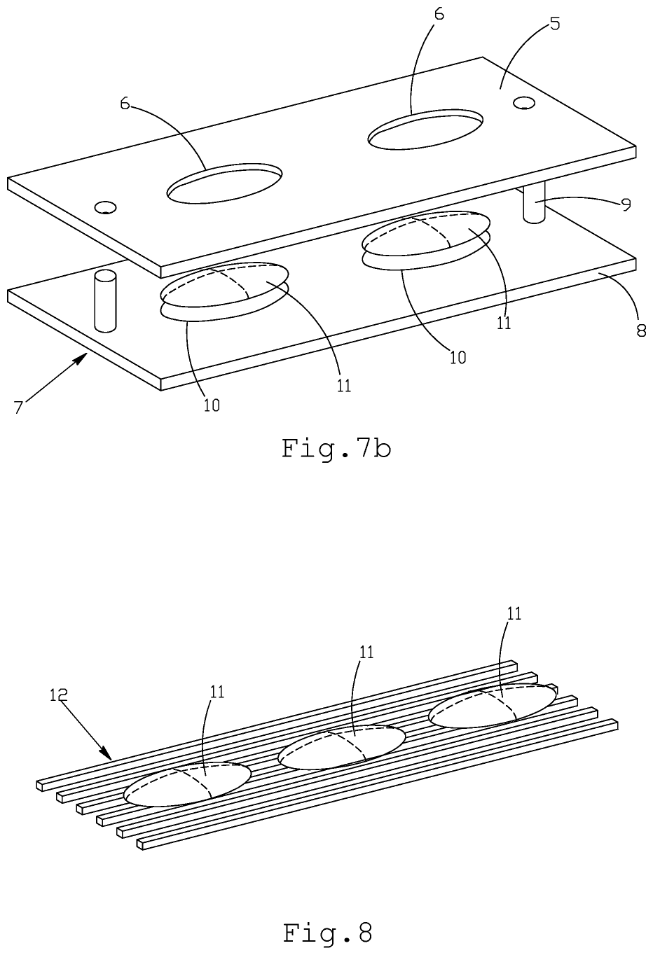

FIGS. 7 to 9 are axonometric views from above of a solid cosmetic product without the pan, at the start of the process according to the present invention.

With reference to the accompanying FIG. 1, the number 100 indicates as a whole the process, comprising the initial step, which is optional.

In FIG. 2 an empty pan 1 is represented. The pan 1 has a circular cross section and is provided with a circular wall 2 with a rim 3 defining an opening 4.

In step R the semi-finished cosmetic product PS in semi-fluid form is introduced into the pan 1 by pouring, until the pan 1 is filled to its rim 3.

Once the pan 1 has been filled completely with the product PS, this is allowed to distribute in the pan and then the product PS is cooled to room temperature (i.e., around 20-25° C.).

In FIG. 3 the pan 1 is represented after it has been filled with the product PS and at the end of the cooling step: the cosmetic product to be completed Pr has a smooth surface S.

At the end of step R, the pan containing the cosmetic product to be completed Pr is placed in a specific container, called pallet (not represented in the figures) with recesses in which to place the pans with the finished cosmetic product to be completed.

After the pan has been placed in the pallet, a specific mask 5 is placed over the opening 4 of the pan (step I), which rests on the rim 3 of the pan 1 (pan 4). In this variant, there is one mask 5 for each single pan 1.

The mask 5 has a perforation 6 corresponding to the type of decoration 8 to be reproduced on the surface S of the cosmetic product to be completed Pr. The perforation of the mask 5 leaves a portion R of the surface S of the cosmetic product to be completed Pr below exposed.

The pallet with the pans 1 with the openings 4 covered by the perforated masks 5 is inserted into an area for depositing the pearlescent pigmented particulate material, in which deposition of pearlescent pigmented particulate material G on the cosmetic product to be completed Pr (step II) (FIG. 5) is performed.

This area is provided with dispersing devices for delivery of the pearlescent pigmented particulate material.

Deposition of the pearlescent pigmented particulate material G is carried out by deposition using pressure. The pressure used is of 6.9 bar.

As can be seen in FIG. 5, said pearlescent pigmented particulate material G deposits on the exposed portion R of the surface S of the cosmetic product to be completed Pr, but also on the perforated mask 5 and on the pallet.

The pearlescent pigmented particulate material G is a mixture of the following pigmented particulate materials: calcium aluminium borosilicate and calcium sodium borosilicate. Said material has particles with dimensions of around 150-200 μm.

The subsequent step (step III) to said step II consists in suctioning the amount of excess pigmented particulate material G that was deposited on the cosmetic product to be completed Pr and on the perforated mask 5. Suctioning is carried out with a suction device that operates with a suction flow rate of 5-35 m³/hour.

The pigmented particulate material G in excess that was removed can thus be collected (step IV) and stored in specific containers (not represented in the figures) for subsequent re-use.

The product obtained at the end of step III is a completed product (FIG. 6), ready to be sent to the packaging site, where the final operations concerning packaging of the completed product in a primary packaging will be carried out.

FIGS. 7a and 7b (FIG. 7b is an exploded view of FIG. 7a) represent the process according to the present invention in the variant that does not provide for the use of the pan, i.e., the finished cosmetic product to be completed has no support. This cosmetic product is in pod form.

In this variant, as in the variant described previously, the pallet 7 has a bottom 8 provided with recesses 10 into each of which to insert the finished cosmetic product without support still to be completed in pods 11. Moreover, supports 9 rise from the bottom 8 of the pallet 7. Said supports form the support base of the mask 5.

In this variant there is only one mask 5, which is in common to several pods. The mask 5 has more than one perforation (in the case illustrated two perforations), each corresponding to each cosmetic product without support to be completed.

FIG. 8 illustrates the finished cosmetic product, without support in pod form 11 still to be completed placed on a grid 12.

1. A completion process for coating a surface of a compact solid cosmetic product obtained by casting with pigmented particulate material G, said process comprising the following steps:
I) positioning a mask on a surface to be completed of said cosmetic product, said mask being suitably perforated so as to leave at least a portion of said surface of said cosmetic product exposed;
II) dispersing a pigmented particulate material G on said exposed surface portion of said cosmetic product, said material being free from liquid carrier;
III) suctioning the pigmented particulate material G deposited on surfaces different from said exposed surface portion of said cosmetic product; and
IV) collecting the pigmented particulate material not deposited on said exposed surface portion of cosmetic product.

2. The process according to claim 1, wherein step (I) is preceded by a step in which a semi-finished cosmetic product is introduced into a secondary packaging having walls defining an opening, the filling of the secondary packaging taking place by pouring.

3. The process according to claim 1, wherein the cosmetic product is placed on a pallet provided with a grid, which is placed inside the pallet so as to cover the internal surface of the bottom of the pallet, said grid being intended to serve as a support base for the cosmetic product to be completed.

4. The process according to claim 1, wherein the mask rests on the pallet or on the walls defining said opening of the secondary packaging.

5. The process according to claim 1, wherein the pigmented particulate material G is delivered at a pressure of between 2 and 10 bars.

6. The process according to claim 1, wherein the pigmented particulate material G is delivered at a pressure of between 6 and 8 bar.

7. The process according to claim 1, wherein the pigmented particulate material G has dimensions ranging from 5 to 500 μm.

8. The process according to claim 1, wherein the pigmented particulate material G has dimensions ranging from 15 to 250 μm.

9. The process according to claim 1, wherein the pigmented particulate material G is pearlescent.

10. A poured cosmetic product obtained by the process according to claim 1 having a surface decorated with pigmented particulate material.
